(19) United States
(12) Patent Application Publication
(10) Pub. No.: US 2023/0081439 A1
(43) Pub. Date: Mar. 16, 2023

(54) GENERATIVE TNA SEQUENCE DESIGN WITH EXPERIMENT-IN-THE-LOOP TRAINING

(71) Applicant: X Development LLC, Mountain View, CA (US)

(72) Inventors: Ryan Poplin, Newark, CA (US); Ivan Grubisic, Oakland, CA (US); Lance Co Ting Keh, La Crescenta, CA (US); Ray Nagatani, San Francisco, CA (US)

(73) Assignee: X Development LLC, Mountain View, CA (US)

(21) Appl. No.: 17/471,903

(22) Filed: Sep. 10, 2021

(51) Int. Cl. G16B 40/20; G16B 20/30; G16B 30/00; G16H 20/00; G06K 9/62

(52) U.S. Cl. CPC G16B 40/20; G16B 20/30; G16B 30/00; G16H 20/00; G06K 9/6256

(57) ABSTRACT

A latent space is defined to represent sequences using training data and a machine-learning model. The training data identifies sequences of molecules and binding-approximation metrics that characterizes whether the molecules bind to a particular target and/or that approximate an extent to which the molecule is more likely to bind to the particular target than some other molecules. Supplemental training data is accessed that identifies other sequences of other molecules and binding affinity scores quantifying binding strengths between the molecules and the particular target. Projections of representations of the other sequences in the supplemental training data are projected in the latent space using the binding affinity scores. An area or position of interest within the latent space is identified based on the projections. A particular sequence represented within or at the area or position of interest or at the position of interest is identified for downstream processing.

### 1. FIELD

The present disclosure relates to using a machine-learning model to generate a latent space representing sequences (e.g., threose nucleic acid sequences) and binding data corresponding a particular target and using the latent space to identify a candidate sequence predicted to bind with the particular target. The latent space can be generated using sequences and binding-approximation metrics and can be subsequently modified using embedded representations of other sequences and binding affinity scores that more precisely characterize binding affinity relative to the binding-approximation metrics.

### 2. BACKGROUND

Accurately predicting binding affinities would provide great advantages across various use cases, such as diagnosing a subject and/or selecting an effective treatment. For example, drug development frequently involves identifying a biological target that is within a particular disease pathway and then attempting to identify a molecule that binds to the biological target. The molecule can itself neutralize function of the target (e.g., by degrading the target) or can serve to link the biological target to another agent (e.g., a cytotoxic agent).

However, each binding affinity is specific to a particular target and a particular molecule. While various high-throughput screens have greatly improved the extent to which data sets can be built to identify various binding variables, these data sets remain incomplete, imprecise in terms of how precisely binding it characterized, or both. Thus, when a given target is identified as being of interest, these data sets can lack sufficient information to identify which molecule(s) will bind to the target with high affinity. Instead, an investigating entity can turn to performing one or more additional high-throughput screens using the target, which can be time-intensive and costly.

It would be advantageous to improve techniques for predicting target-specific binding affinities and/or for predicting which molecule(s) will bind to a specific target with high affinity.

### 3. BRIEF SUMMARY

In some embodiments, a computer-implemented method is provided. Training data that includes a set of training data elements is accessed. Each of the set of training data elements identifies a sequence of a molecule and a binding-approximation metric that characterizes whether the molecule binds to a particular target and/or that approximates an extent to which the molecule is more likely to bind to the particular target than other molecules associated with at least some other sequences. A latent space for representing sequences is defined by processing the training data using a machine-learning model. Supplemental training data is accessed that includes a set of supplemental training elements, each of the set of supplemental training data elements identifying a sequence of a molecule and a binding affinity score that quantifies a strength of a binding interaction between the molecule and the particular target. Representations of the sequences are projected in the supplemental training data in the latent space using the binding affinity scores to generate an updated latent space. An area of interest within the latent space or a position of interest within the latent space is identified based on binding affinity scores of the supplemental training data and positions of the projected representations of the sequences represented in the supplemental training data within the latent space. A particular sequence represented within the area of interest or at the position of interest is identified. A determination of a particular binding affinity score of the particular sequence with the particular target using an in vitro experiment is facilitated.

Identifying the area of interest within the latent space or the position of interest within the latent space can include identifying a starting position within the latent space; and identifying a direction from the starting point associated with a gradient in binding affinity scores. The area of interest within the latent space or the position of interest within the latent space can be identified using the starting position and the direction.

The particular sequence can be a threose nucleic acid sequence.

Identifying the area of interest within the latent space or the position of interest within the latent space can include defining a separating hyperplane using another machine-learning model.

The method can further include projecting a representation of the particular sequence in the updated latent space using the particular binding affinity; identifying a new area of interest within the latent space or a new position of interest within the latent space based on the particular binding affinity score and a position of the projected representation of the particular location in the latent space; identifying a different particular sequence represented within the new area of interest or at the new position of interest; and facilitating determining a new particular binding affinity score for the different particular sequence and the particular target using an in vitro experiment.

The machine-learning model can include a generator model.

The machine-learning model includes a variational autoencoder model.

The machine-learning model can include a ResNet model.

The latent space can include at least 12 dimensions.

Defining the latent space can be based on the sequences of the molecules and the binding-approximation metrics in the training data.

The training data can have been generated using a Systematic Evolution of Ligands by Exponential Enrichment (SELEX) technique.

The supplemental training data can have been generated using a Biolayer Inferometry Analysis (BLI) technique.

The method may further include updating a data store to store the particular binding affinity score in association with the particular sequence, where the data store associates various molecules (e.g., various aptamers) with corresponding binding affinity scores that characterize binding affinities with the particular target.

The method may further include selecting a specific sequence using the data store, wherein a selection condition is satisfied for the specific sequence; and outputting an identification corresponding to the specific sequence.

The method may further include: outputting an identification of a potential treatment for a given medical condition or for a subject with the given medical condition, wherein the potential treatment includes molecules coded by the particular sequence.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

A better understanding of the nature and advantages of embodiments of the present invention can be gained with reference to the following detailed description and the accompanying drawings.

### 4. BRIEF DESCRIPTION OF THE DRAWINGS

FIG. 1 shows an exemplary network for selecting one or more sequences using latent space representations and machine learning.

FIG. 2 shows a block diagram of an exemplary binding-approximation metric collection system according to some embodiments of the invention.

FIG. 3 is a flowchart of an example process to identify a particular sequence predicted to code for a molecule to bind (e.g., at least with a threshold absolute or relative affinity) with a particular target.

FIG. 4 illustrates a measurement system according to some embodiments of the present invention.

FIG. 5 shows an exemplary computing device in accordance with some embodiments of the present invention.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### 5. DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes can be made in the function and arrangement of elements without departing from the spirit and scope as set forth in the appended claims.

Specific details are given in the following description to provide a thorough understanding of the embodiments. However, it will be understood that the embodiments can be practiced without these specific details. For example, circuits, systems, networks, processes, and other components can be shown as components in block diagram form in order not to obscure the embodiments in unnecessary detail. In other instances, well-known circuits, processes, algorithms, structures, and techniques can be shown without unnecessary detail in order to avoid obscuring the embodiments.

Also, it is noted that individual embodiments can be described as a process which is depicted as a flowchart, a flow diagram, a data flow diagram, a structure diagram, or a block diagram. Although a flowchart or diagram can describe the operations as a sequential process, many of the operations can be performed in parallel or concurrently. In addition, the order of the operations can be re-arranged. A process is terminated when its operations are completed, but could have additional steps not included in a figure. A process can correspond to a method, a function, a procedure, a subroutine, a subprogram, etc. When a process corresponds to a function, its termination can correspond to a return of the function to the calling function or the main function.

### I. NETWORK FOR SELECTING SEQUENCES USING LATENT SPACE REPRESENTATIONS AND MACHINE LEARNING

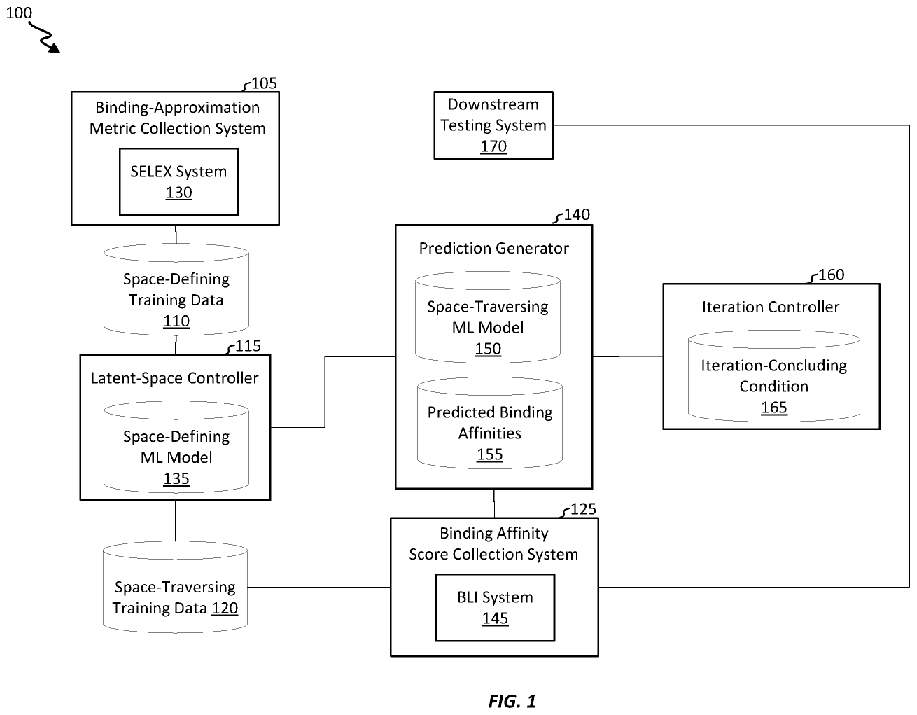

FIG. 1 shows an exemplary network 100 for selecting one or more sequences using latent space representations and machine learning. More specifically, machine-learning techniques use multiple types of training data that characterize molecules' binding affinities with a particular target to generate and/or traverse a latent space. The latent space includes a compressed representation of the training data and can have dimensions defined to emphasize important and/or semantically interesting features of the training data. The latent space can be used to predict a binding affinity of another molecule with the particular target and/or identify one or more candidate molecules predicted to bind with high affinity to the particular target.

More specifically, a first part of a training data set can include space-defining training data 110 can be received by a latent-space controller 115 from a binding-approximation metric collection system 105. A second part of the training data set can include space-traversing training data 120 be received by latent-space controller 115 from a binding affinity score collection system 125. Space-defining training data 110 can be received from binding-approximation metric collection system 105 in response to a request sent by latent-space controller to binding-approximation metric collection system 105 (e.g., that identifies a type of sequence of interest and/or one or more other sequence properties). Space-traversing training data 120 can be received from binding affinity score collection system 125 in response to a request sent by latent-space controller 115 to binding affinity score collection system 125 (e.g., that identifies a type of sequence of interest and/or one or more other sequence properties).

Space-defining training data 110 includes multiple training data elements, each of which includes or is associated with a sequence. Similarly, space-traversing training data 120 includes multiple training data elements, each of which includes or is associated with a sequence. The sequences associated with space-defining training data 110 and/or the sequences associated with space-traversing training data 120 can include (for example) a threose nucleic acid (TNA) sequence, a deoxyribonucleic acid (DNA) sequence, a ribonucleic acid (RNA) sequence, or a xeno nucleic acid sequence (XNA). TNA is an artificial genetic polymer where a four-carbon threose sugar replaces the naturally occurring five-carbon ribose sugar in RNA, which prevents the polymer for being subject to nuclease digestion.

Each sequence included in or associated with a training data element in space-defining training data 110 and/or space-traversing training data 120 can code for a molecule, such as an aptamer, oligonucleotide, small molecule, large molecule, or polypeptide. In some instances, each training data element in space-defining data 110 and each training data element in space-traversing training data 120 includes or is associated with a TNA sequence that codes for a corresponding aptamer molecule.

Aptamers are short sequences of single-stranded oligonucleotides (e.g., anything that is characterized as a nucleic acid, including xenobases). The sugar backbone of the single-stranded oligonucleotides functions as the acid and the A (adenine), T (thymine), C (cytosine), G (guanine) refers to the base. An aptamer can involve modifications to either the acid or the base. Aptamers have been shown to selectively bind to specific targets (e.g., proteins, protein complexes, peptides, carbohydrates, inorganic molecules, organic molecules such as metabolites, cells, etc.) with high binding affinity.

As compared to antibodies, aptamers are relatively inexpensive to produce, relatively stable in select environments (e.g., that include proteases or enzymes that can degrade antibodies or in low-pH environments like the gut), and are relatively small to facilitate traversing biological borders, such as the Blood-Brain Barrier). Further, aptamers can be highly specific, in that a given aptamer can exhibit high binding affinity for one target but low binding affinity for many other targets. Thus, aptamers can be used to (for example) bind to disease-signature targets to facilitate a diagnostic process, bind to a treatment target to effectively deliver a treatment (e.g., a therapeutic or a cytotoxic agent linked to the aptamer), bind to target molecules within a mixture to facilitate purification, bind to a target to neutralize its biological effects, etc. However, the utility of an aptamer hinges on a degree to which it effectively binds to a target.

Aptamers are also able to be sequenced quickly to determine the identity of an aptamer that successfully bound to a given target (and therefore the cell receptors that it binds, and the genetic identity of the target). While aptamers have an advantage of having high specificity and high sensitivity (and relatively low costs/high speed of manufacture compared to, for example, monoclonal antibodies in most diagnostic applications), the high sensitivity of aptamers across targets means that a given aptamer that binds with very high affinity to one target typically binds poorly with the vast majority of other targets. Thus, predicting which aptamer will bind with high affinity to any particular target has been traditionally challenging.

Each training data element in space-defining training data 110 further includes a binding-approximation metric that indicates whether a molecule coded for by the sequence included in or associated with the training data element bound to a particular target. The binding-approximation metric can include (for example) a binary value or a categorical value. The binding-approximation metric can indicate whether the molecule bound to the particular target in an environment where the molecule and other molecules (e.g., other potential binders) are concurrently introduced to the particular target.

The binding-approximation metric can be determined using a first assay, such as Systematic Evolution of Ligands by EXponential Enrichment (SELEX). SELEX is an iterative experimental process where a nucleic acid library of oligonucleotide strands (aptamers) is incubated with a target molecule. Then, the target-bound oligonucleotide strands are separated from the unbound strands and amplified via polymerase chain reaction (PCR) to seed a new pool of oligonucleotide strands. This selection process is continued for a number (e.g., 6-15) rounds with increasingly stringent conditions, which ensure that the oligonucleotide strands obtained have the highest affinity to the target molecule.

The first assay can be performed using one or more pieces of laboratory equipment. For example, SELEX can be performed at or using a SELEX system 130, which can include one or more pieces of equipment to facilitate performing part or all of SELEX semi-automatically or automatically. Exemplary equipment could include an incubator, PCR machine (or GeThermocycler), Gel Electrophoresis equipment, and/or a device for DNA quantification (e.g., a Nanodrop). While FIG. 1 shows SELEX system 130 as being part of binding-approximation metric collection system 105, it will be appreciated that in some other embodiments, SELEX system 130 can be remote from binding-approximation metric collection system 105 (but can communicate binding-approximation metrics corresponding to sequences to binding-approximation metric collection system 105). Further, it will be appreciated that binding-approximation metric collection system 105 can include and/or communicate with multiple SELEX systems 130 so as to collect binding-approximation metrics from multiple data sources.

While SELEX data is an example data source that can include data identifying binding-approximation metrics for a relatively large number of sequences (e.g., 10¹⁴-10¹⁵ TNA sequences corresponding to an individual target), this data collection still is a very small fraction of the total TNA sequences that may exist (e.g., 10²⁴ for a 40-nucleotide aptamer). Thus, it is unlikely that the SELEX data includes binding-approximation metrics corresponding to sequences that most tightly bind to a target of interest.

Thus, a latent-space controller 115 uses the space-defining training data 110 to train and use a space-defining machine learning model 135 to define a latent space for the particular target. The latent space can relate embedded representations of sequences to binding-approximation metrics. An embedded representation of the sequence can include one-hot encoding of each nucleotide in the sequence that maintains information about the order of the nucleotides. The representation of the sequence can additionally or alternatively include other features concerning the sequence and/or a molecule coded for by the sequence, for example, post-translational modifications, binding sites, enzyme active sites, local secondary structure, kmers or characteristics identified for specific kmers, etc. The latent space can include one or more dimensions (e.g., at least 5 dimensions, at least 8 dimensions, at least 10 dimensions, or at least 15 dimensions) that—for a given sequence—represent the sequence and another dimension that represents the corresponding binding-approximation metric. Space-defining machine learning model 135 can be configured (e.g., via a loss function or objective function) to define one or more dimensions of the latent space to prioritize smooth and/or monotonic changes in binding-approximation metrics across the dimension. For example, the one or more dimensions can be defined to optimize a likelihood of representations of sequences corresponding to highest binding affinities (or highest binding-approximation metrics) being positioned at or near an extreme of each of the dimensions or at or near an origin of the dimensions.

Space-defining machine learning model 135 can include an encoder network and a decoder network. The encoder network and/or the decoder network can include a ResNet model. The encoder network and/or the decoder network can include at least 5 layers, at least 10 layers, at least 25 layers, at least 50 layers, at least 75 layers, at least 100 layers, at least 125 layers, or at least 150 layers. The encoder network and/or the decoder network can include a skip connection to connect non-sequential layers. The machine-learning model can include a Generative Adversarial Network (GAN), a conditional GAN, a Variational Autoencoder, or a clustering algorithm (e.g., that uses a component analysis, such as principal component analysis or independent component analysis). More specifically, the GAN or Variational Autoencoder can be used to train a decoder network within the model to reconstruct raw data (e.g., a binding-approximation metric) based on latent-space information (e.g., a position within the latent space).

After the latent space is defined using space-defining training data 120, latent-space controller 115 can enhance the latent space with space-traversing training data 120. As noted above, each training data element in space-traversing training data 120 includes or is associated with a sequence. Further, each training data element in space-traversing training data 120 includes a binding affinity score that quantify a strength of a binding interaction between a molecule coded by the sequence and the particular target. Enhancing the latent space with space-traversing training data 120 can include projecting representations of (e.g., embedded representations of) sequences in the training data elements and of binding affinity scores in the latent space.

The binding affinity scores in space-traversing training data 120 can be more accurate, more precise, and/or more standardized as compared to the binding-approximation metrics in space-defining training data 110. The binding affinity scores can be determined using a second assay or by retrieving results from a data store where the results had been generated using a second assay. The second assay can include Bio-Layer Interferometry (BLI). In this context, BLI includes preparing a bionsensor tip to include the multiple molecules of a type coded for by a given sequence in an immobilized form and a solution with the particular target in a tip of a biosensor and an internal reference number. Binding between the molecule(s) and the particular target increases a thickness of the tip off the biosensor. The biosensor is illuminated using white light, and an interference pattern is detected. The interference pattern and temporal changes to the interference pattern (relative to a time at which the molecules and particular target are introduced to each other) are analyzed to predict binding-related characteristics, such as binding affinity, binding specificity, a rate of association, and a rate of dissociation.

The second assay can be performed using one or more pieces of laboratory equipment. For example, BLI can be performed at or using a BLI system 145, which can include one or more pieces of equipment to facilitate performing part or all of BLI semi-automatically or automatically. Exemplary equipment includes a BLI instrument and/or a device to measure protein concentration (e.g., a spectrophotometer). While FIG. 1 shows BLI system 145 as being part of binding affinity score collection system 125, it will be appreciated that in some other embodiments, BLI system 145 can be remote from binding affinity score collection system 125 (but can communicate binding affinity scores corresponding to sequences to binding affinity score collection system 125). Further, it will be appreciated that binding affinity score collection system 125 can include and/or communicate with multiple BLI systems 145 so as to collect binding-approximation metrics from multiple data sources.

A precision and/or accuracy of the binding affinity scores can be better than (e.g., more precise and/or more accurate relative to) a precision and/or accuracy of the binding-approximation metrics (e.g., due to a difference in the assays and/or experiments used to generate the binding affinity scores and binding —approximation metrics).

In some instances, binding affinity scores and binding-approximation metrics are numeric values on a same scale or are categorical values on a same scale. In some instances, a precision of binding affinity scores is higher than a precision of binding-approximation metrics. For example, binding-approximation metrics can be binary numbers (0 or 1), and binding affinity scores can be real values on a scale of 0 to 1. In some instances, pre-processing is performed to binding-approximation metrics or to binding affinity scores so as to bring values onto a same scale.

Space-traversing training data 120 can include sequences associated with binding-affinity scores representing poor (or no) binding affinity to the particular target in addition to binding affinity scores representing one or more binding affinity scores representing moderate or strong binding affinity to the particular target. Even the data corresponding to poor binders can facilitate defining the latent space, such that useful predictions can be generated. For each sequence in space-traversing training data 120, latent-space controller 115 projects a representation (e.g., embedding of) of the sequence and the corresponding binding affinity score into the latent space.

For each of one or more positions or regions of the latent space, a prediction generator 140 can use the latent space to generate a predicted binding affinity 155 (e.g., a predicted binding affinity score) for the position or region. In some instances, prediction generator 140 can generate a predicted binding affinity 155 for each of multiple, many, or all positions or regions in the latent space.

In some instances, prediction generator 140 can use the latent space, space-traversing training data 120 and another machine learning model to facilitate generating predicted binding affinity scores and/or identifying the one or more sequences that correspond to high predicted binding affinity scores. The other machine learning model may include (for example) a support vector machine or a regression model (e.g., a linear regression model).

Prediction generator 140 can then use the predicted binding affinities, the latent space and/or the projections to identify one or more sequences (corresponding to one or more positions within the latent space and/or one or more regions within the latent space) that correspond to high predicted binding affinity scores.

In some instances, the identification of the one or more sequences can be based on:
the relative positions within the latent space of the projections of some or all of space-traversing training data 120 compared to each other;
the relative positions within the latent space of the projections of some or all of space-traversing training data 120 and space-defining training data 110 compared to each other; and/or
one or more gradients of the latent space.

In some instances, prediction generator 140 can use a space-traversing machine learning model 150 to characterize how binding affinity scores vary across directions in the space and/or to identify one or more sequences of interest. By traversing and characterizing this latent space, prediction generator 140 can identify one or more select sequences associated with high predicted binding affinity with the particular target. That is, each of one or more select sequences can be associated with a predicted binding affinity corresponding to a prediction that a molecule coded by the sequence will bind to the particular target, will bind sufficiently strongly to the particular target, or will bind with at least a threshold binding affinity with the particular target.

In some instances, the latent space can be used to determine a position for each of the data points in the space-traversing training data 120 where it is to be embedded, and the positions and corresponding binding affinity scores associated with the data points can be used to identify the one or more sequences predicted to correspond to high affinity scores (by generating predicted binding affinities 155). Gradients of the latent space can further be used to determine the one or more sequences of interest.

For example, prediction generator 140 can generate one or more seed locations (e.g., using a random or pseudo-random protocol), or prediction generator 140 can identify one or more seed sequences (e.g., using a random or pseudo-random protocol) that can then be used to identify one or more seed locations. From each seed location, prediction generator 140 can use space-traversing machine learning model 145 to define a vector that extends along a direction towards a large delta or largest delta in predicted binding affinity score 155s (e.g., using space-traversing machine learning model 150 and the definition of the latent space). In some instances, the vector is identified by defining the vector such that it has a large slope in binding affinity scores. In some instances, the vector is identified by prioritizing defining the vector such that an endpoint of the vector corresponds to a large predicted binding affinity score (or large predicted binding affinity score). The vector can be identified by using space-traversing machine learning model 150 to identify a hyperplane that separates sequences corresponding to molecules that bound to the target molecule from sequences corresponding that molecules that did not bind to the target molecule. The vector can be identified by using space-traversing machine learning model 150 to identify a hyperplane that separates sequences corresponding to molecules that bound with at least a threshold affinity to the target molecule from sequences corresponding that molecules that did not bind to the target molecule with at least the threshold affinity.

The vector can be used to identify a portion or position within the feature space associated with one or more highest predicted binding affinities in association with the particular target. Each of one or more sequences represented within the portion or the sequence represented at the position can then be characterized as corresponding to a sequence of interest (e.g., a potential high-affinity binder candidate).

As another example, prediction generator 140 can generate predicted binding affinities 155 across a multi-dimensional grid of the sequence-related dimensions and identifies one or more local or absolute maxima of predicted binding-affinity scores 155. Prediction generator 140 can then characterize sequences corresponding to the local or absolute maxima as sequences of interest.

As another example, space-traversing machine learning model 150 includes a greedy search algorithm to determine how to navigate the latent space and to identify one or more sequences of interest.

As yet another example, space-defining machine learning model 135 can be configured to define clusters within the latent space, and space-traversing machine learning model 150 can be configured to navigate through the latent space to identify one or more clusters of interest. Prediction generator 140 may then identify sequences within one or more sub-spaces of the latent space that correspond to the clusters of interest, and those identified sequences can be characterized as sequences of interest.

Prediction generator 140 can avail or transmit each of one, more or all of the sequences of interest to binding affinity score collection system 125 to request determining a binding affinity score of a molecule coded for by each of the one, more or all of the sequences of interest with respect to the particular target (e.g., using BLI system 145). Binding affinity score collection system 125 can then determine a binding affinity score for each of the one, more or all of the sequences of interest and can return then as additional space-traversing training data 120 to latent-space controller so project the new binding affinity scores and corresponding sequence representations in the latent space. Each of the binding affinity scores can include an equilibrium dissociation constant (Ka), an association rate, and/or a dissociation rate, which can facilitate obtaining these binding affinity scores rather quickly (e.g., as compared to precisely measuring binding). In instances where a binding affinity score includes two or more of: an equilibrium constant, an association rate, and a dissociation rate, the variables can be independent incorporated into the latent space.

In some instances, prediction generator 140 then projects representations of the sequences of interest are onto the latent space. Prediction generation 140 can the identify one or more new sequences of interest using space-traversing machine learning model 150 and the latent space (e.g., by redefining a hyperplane or vector that characterizes modulations of binding affinity scores across the space).

Iterations of validation actions including (1) updating the latent space with new known (e.g., experimentally identified) binding affinity scores, (2) identifying one or more new sequences of interest for which binding affinity scores are to be collected, and (3) determining the binding affinity scores can be repeated across multiple rounds until an iteration controller 160 determines that an iteration-concluding condition 165 is satisfied. Thus, each time an iteration cycles, space-traversing training data 120 grows based on a previous iteration and is used to update the latent space. Iteration-concluding condition 165 can be defined to be satisfied when (for example):
at least a threshold number of rounds (e.g., one, at least one, at least three, at least five, at least ten, etc.) are completed;
an experimental determination of a binding affinity score of a molecule encoded by at least one new sequence of interest is above a predefined threshold,
a percentage increase in a maximum binding affinity score across the molecule(s) encoded by a round's new sequence(s) of interest relative to a most-recent round's maximum binding affinity score is below a predefined threshold;
a difference between a maximum binding affinity score across the molecule(s) encoded by a round's new sequence(s) of interest relative to a most-recent round's maximum binding affinity score is below a predefined threshold;
a percentage increase in a statistic of binding affinity scores for the molecule(s) encoded by a round's new sequence(s) of interest relative to a most-recent round's corresponding statistic is below a predefined threshold; or
a difference between a statistic of binding affinity scores for the molecule(s) encoded by a round's new sequence(s) of interest relative to a corresponding statistic of a most-recent round's maximum binding affinity score is below a predefined threshold.

It will be appreciated that multiple iteration-concluding conditions 165 can be evaluated. For example, iteration-concluding condition 165 can be satisfied when: (1) at least a predefined threshold rounds of the set of validation actions were performed; (2) for a given molecule—the binding affinity with the particular molecule exceeds a binding-affinity threshold; and (3) a probability of observing a severe adverse event (as determined based on across-subject or event-frequency data) is less than an adverse-event threshold when a composition including the molecule was administered.

In some instances, an iteration-concluding condition 165 (e.g., of multiple iteration-concluding conditions 165 that can be evaluated to determine whether to conclude the iterations) is configured to be satisfied upon receiving an input from a user that corresponds to an instruction or confirmation for the iterations to be concluded. For example, an interface can be transmitted to or presented at a user device that identifies one or more of: an iteration round; a binding affinity score of each of one or more molecules encoded by the most recent round's one or more new sequences of interest; the most recent round's one or more new sequences of interest; a previous round's one or more sequences of interest; a binding affinity score of each of one or more previous round's one or more sequences of interest; etc. Input by the user can correspond to an instruction to proceed with downstream testing for each sequence identified via the interface or identified as a sequence of interest in a most-recent iteration. Alternatively, the user input can include a selection of an incomplete subset of the sequences identified within the interface or an incomplete subset of the sequences of interest identified in a most-recent iteration. The interface can further be configured to allow the user to provide input that identifies one or more additional sequences and/or one or more additional molecules for downstream testing.

When iteration controller 160 determines that iteration-concluding condition is satisfied, a trigger can be sent to a downstream testing system 170. The trigger can identify a sequence associated with the satisfaction of the iteration-concluding condition(s) 165 or a molecule coded by a sequence associated with satisfaction of iteration-concluding condition(s) 165.

The trigger can cause an experiment-instruction communication to be sent to another device and/or other system, and the communication can identify the sequence(s) and/or molecule(s) associated with satisfaction of the condition. An experiment can include producing (generating or accessing) each of the identified molecules and/or the molecule(s) coded by the identified sequence(s). The experiment can include validating the molecules in a wet lab in either individual or bulk experiments.

The trigger can initiate a different kind of assessment of the sequence or molecule, in vitro testing to further predict an impact of the molecule on a particular medical condition (e.g., with regard to disrupting a pathway of the particular medical condition or with regard to slowing a progression of the particular medical condition, stopping progression of the particular medical condition, or curing the particular medical condition), in vivo testing to further predict an impact of the molecule on a particular medical condition, manufacture of a composition that includes the molecule, and/or administration of a composition that includes the molecule to a subject. The different kind of assessment can be initiated by, monitored by, and/or performed by downstream testing system 170.

In some instances, the trigger results in or facilitates manufacture and/or use of a pharmaceutical composition that includes the molecule coded by a sequence associated with satisfaction of iteration-concluding condition 165. The pharmaceutical composition can further include a pharmaceutically acceptable carrier and/or a surfactant. The pharmaceutical composition can be administered to a subject (e.g., a human subject) via (for example) an oral, intravenous, intramuscular, intranasal, or intradermal route. The human subject can have been diagnosed with a particular medical condition associated with a particular medical pathway that involves the particular for which the latent space was defined and used. Administration of the pharmaceutical composition can be used to (for example) facilitate a diagnosis of a medical condition, treat a medical condition, or neutralize a biological effect of a given previously administered treatment.

In some instances, rather than defining and/or navigating a latent space only based on binding variables, the latent space may be defined and/or may be navigated using one or more other variables in addition to or instead of a binding variable. For example, a latent space may be defined to include multiple dimensions that represent individual sequences, another dimension that represents a binding variable, and yet another dimension that represents a stability variable. The space may be navigated using additional data that corresponds to binding and/or stability measurements of additional sequences.

Various embodiments disclosed herein include distinct novel characteristics and specific technical advantages. For example, some embodiments include defining a latent space based on binding-associated results corresponding to a first assay but determining how to traverse the latent space based on binding-associated results corresponding to a second assay, where results from the first assay can be less precise and/or less accurate as compared to results from the second assay. While it can seem non-intuitive to define a latent space using less accurate or less precise data, the inventors recognized that this approach can provide a larger quantity of data and that a high quantity of data is particularly important for defining a latent space (as compared to a smaller yet more accurate and/or more precise data set). Meanwhile, it can be more advantageous to capitalize on more accurate and/or more precise data while identifying particular regions or particular positions of latent space (corresponding to one or more sequence representations).

As another example, some embodiments relate to using a sophisticated machine-learning model to define a latent space, and some (same or different embodiments) include building and/or using a high-dimensional latent space (e.g., having at least three dimensions representing sequences, having at least five dimensions representing sequences, having at least ten dimensions representing sequence, having at least twelve dimensions representing dimensions, having at least four total dimensions, having at least six dimensions, having at least eleven dimensions, or having at least thirteen dimensions). Complications with using a sophisticated model and dealing with a high-dimensional space is that if a training data set that is used to define the space too small or is not sufficiently representative of the variability of potential sequences, the field can be defined in a manner that does not sufficiently capture relationships between sequences (e.g., in terms of capturing which features of sequences correspond to particularly high or particularly low binding affinity with the particular target) and/or does not capture sufficiently dependencies between sequence features and binding affinity with the particular target. A high-dimensional latent space can also facilitate identifying a sequence that satisfies a given iteration-concluding condition after a relatively short time and/or with a relatively small number of iterations. For example, if the latent space is sufficiently sophisticated, it can be possible that a very first round of predictions generated based on a first accessed supplemental training data set or within few iterations.

The efficiency of techniques disclosed herein is compounded by the fact that the techniques and data set are sufficiently powerful to be able to be trained on a very inclusive set of data. Frequently, when a model is to be trained and an input data space is very large, a pre-processing step involves focusing on an area of the input space of interest. For example, a pre-processing step can include identifying a subset of an initial training data supraset for which binding affinity metrics indicated corresponding molecules experienced at least some level of binding with the particular target. A latent space can then be defined and explored to predict which sequence corresponds to a highest binding affinity. Meanwhile, various approaches disclosed herein facilitate training using a large data set, using a complex model, and complementing the initial training with more precise data that facilitates cycles of focused data collection. In these circumstances, defining the latent space based on data both from sequences corresponding to molecules that bound to the particular target and also molecules that did not bind to the particular target facilitates accurately characterizing the space.

Further, some embodiments relate to using and selecting TNA sequences of aptamers. Aptamers are particularly advantageous in a protocol that involves iterating between generating model outputs, collecting experimental data corresponding to model predictions, and updating the model for multiple reasons. For example, as compared to antibodies, aptamers are relatively inexpensive to produce and can be produced quickly. Further, physiologically, antibodies can access targets that antibodies generally cannot in subjects, such as targets in the brain (as antibodies cannot pass the blood-brain barrier), targets in the gut (as antibodies are unstable in low-pH environments), and targets in or near solid tumors (as antibodies are more susceptible to proteases secreted that aid in metastasis).

### II. EXEMPLARY BINDING-APPROXIMATION METRIC COLLECTION SYSTEM

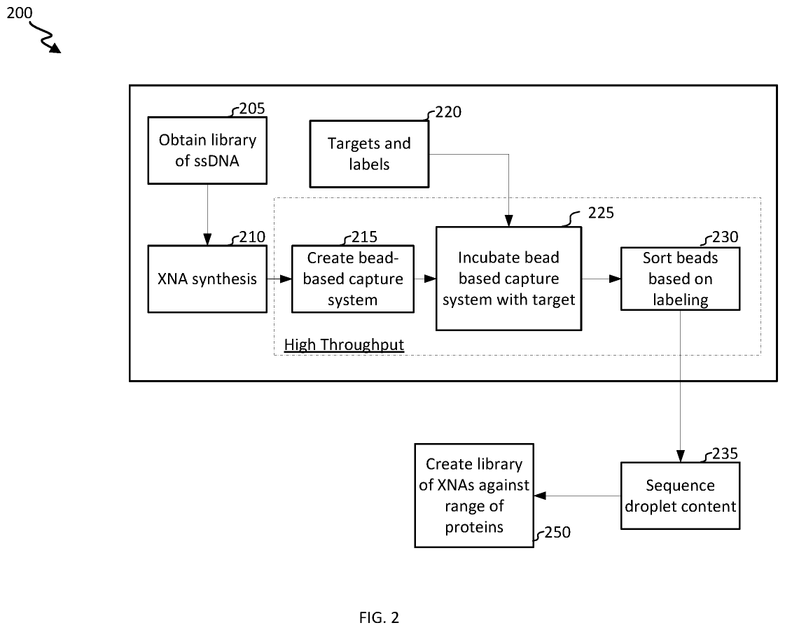

FIG. 2 shows a block diagram of an exemplary binding-approximation metric collection system 200. It will be appreciated that binding-approximation metric collection system 105 and/or SELEX system 130 can include one or more components and/or one or more characteristics of binding-approximation metric collection system 200 and/or can perform one or more operations of binding-approximation metric collection system 200.

Binding-approximation metric collection system 200 can facilitate identifying particular aptamers for in vitro experiments to assess queries, such as binding affinities or product inhibition with respect to one or more particular targets. In various embodiments, binding-approximation metric collection system 200 implements screening-based techniques for aptamer discovery where each aptamer candidate sequence in a library is assessed based on the query (e.g., binding affinity with one or more targets or functionally capable of inhibiting one or more targets) in a high-throughput manner.

In some embodiments, binding-approximation metric collection system 200 implements machine learning based techniques for enhanced aptamer discovery where each aptamer candidate sequence in a library that satisfies the query is input into one or more machine-learning models to predict additional aptamer candidate sequences that potentially satisfy the query. In some embodiments, the binding-approximation metric collection system 200 further implements screening-based techniques for aptamer validation to validate or confirm that the predicted additional aptamer candidate sequences do satisfy the query (e.g., bind or inhibit the one or more targets). For example, downstream testing system 170 from network 100 (depicted in FIG. 1) can include part or all of binding-approximation metric collection system 100. As should be understood, these techniques from screening through prediction to validation can be repeated in one or more closed loop processes sequentially or in parallel to ultimately assess any number of queries in a high through-put manner.

Binding-approximation metric collection system 200 includes obtaining one or more single stranded DNA (deoxyribonucleic acid) or RNA (ribonucleic acid) (ssDNA [single-stranded DNA] or ssRNA [single-stranded RNA]) libraries at block 205. The one or more ssDNA or ssRNA libraries can be obtained from a third party (e.g., an outside vendor) or can be synthesized in-house, and each of the one or more libraries typically contains up to 10¹⁷ different unique sequences.

At block 210, the ssDNA or ssRNA of the one or more libraries are transcribed to synthesize a Xeno nucleic acid (XNA) aptamer library. XNA aptamer sequences (e.g., threose nucleic acids [TNA], 1,5-anhydrohexitol nucleic acid [HNA], cyclohexene nucleic acid [CeNA], glycol nucleic acid [GNA], locked nucleic acid [LNA], peptide nucleic acid [PNA], FANA [fluoro arabino nucleic acid]) are synthetic nucleic acid analogues that have a different sugar backbone than the natural nucleic acids DNA and RNA. XNA can be selected for the aptamer sequences, as these polymers are not readily recognized and degraded by nucleases, and thus they are well-suited for in vivo applications. XNA aptamer sequences can be synthesized in vitro through enzymatic or chemical synthesis. For example, an XNA library of aptamers can be generated by primer extension of some or all of the oligonucleotide strands in a ssDNA library, flanking the aptamer sequences with fixed primer annealing sites for enzymatic amplification, and subsequent PCR amplification to create an XNA aptamer library that includes 10¹²- 10¹⁷ aptamer sequences.

In some instances, the aptamer sequences can be processed to generate initial sequence data comprising a representation of the sequence of each aptamer and optionally a count metric. The representation of the sequence can include an embedding, such as a one-hot encoding of each nucleotide in the sequence, where the embedded representation maintains information about the order of the nucleotides in the aptamer. The representation of the sequence can additionally or alternatively include a string of category identifiers, with each category representing a particular nucleotide. The count metric can include a count of each aptamer in the XNA aptamer library.

At block 215, the aptamers within the XNA aptamer library are partitioned into monoclonal compartments (e.g., monoclonal beads or compartmentalized droplets) for high-throughput aptamer selection. For example, the aptamers can be attached to beads to generate a bead-based capture system for a target. Each bead can be attached to a unique aptamer sequence generating a library of monoclonal beads. The library of monoclonal beads can be generated by sequence-specific partitioning and covalent attachment of the sequences to the beads, which can be polystyrene or glass beads. In some instances, the sequence-specific partitioning includes hybridization of XNA aptamers with capture oligonucleotides having an amine modified nucleotide for interaction with covalent attachment chemistries coated on the surface of a bead. In certain instances, the covalent attachment chemistries include N-hydroxysuccinimide (NHS) modified PEG, cyanuric chloride, isothiocyanate, nitrophenyl chloroformate, hydrazine, or any combination thereof.

At block 220, a target (e.g., proteins, protein complexes, peptides, carbohydrates, inorganic molecules, cells, etc.) is obtained. The target can be obtained as a result of query posed by a user (e.g., a client or customer). For example network 100 from FIG. 1 can initiate processing to generate predicted binding affinities 155 and/or coordinate downstream testing of one or more select sequences in response to receiving a user request to identify one or more aptamers that bind with high affinity to a particular target and/or one or more sequences the code for one or more aptamers that bind with high affinity to a particular topic. The user request can specify the particular target. Latent-space controller 115 can thereafter use the identification of the particular target when requesting and/or retrieving space-defining training data 110 from binding-approximation collection system 105.

At block 225, the bead-based capture system is incubated with the labeled target to allow for the aptamers to bind with the target and form aptamer-target complexes.

At block 230, the beads having aptamer-target complexes are separated from the beads having non-binding aptamers using a separation protocol. In some instances, the separation protocol includes a fluorescence-activated cell sorting system (FACS) to separate the beads having the aptamer-target complexes from the beads having non-binding aptamers. For example, a suspension of the bead-based capture system can be entrained in the center of a narrow, rapidly flowing stream of liquid. The flow can be arranged so that there is separation between beads relative to their diameter. A vibrating mechanism causes the stream of beads to break into individual droplets (e.g., one bead per droplet). Before the stream breaks into droplets, the flow passes through a fluorescence measuring station where the fluorescent label which is part of the aptamer-target complexes is measured. An electrical charging ring can be placed at a point where the stream breaks into droplets. A charge can be placed on the ring based on the prior fluorescence measurement, and the opposite charge is trapped on the droplet as it breaks from the stream. The charged droplets can then fall through an electrostatic deflection system that diverts droplets into containers based upon their charge (e.g., droplets having beads with aptamer-target complexes go into one container and droplets having beads with non-binding aptamers go into a different container). In some instances, the charge is applied directly to the stream, and the droplet breaking off retains a charge of the same sign as the stream. The stream can then returned to neutral after the droplet breaks off

At block 235, the aptamers from the aptamer-target complexes are eluted from the beads and target, and amplified by enzymatic or chemical processes to optionally prepare for subsequent rounds of selection (repeat blocks 210-230, for example a SELEX protocol). The stringency of the elution conditions can be increased to identify the tightest-binding or highest affinity sequences. In some instances, once the aptamers are separated and amplified, the aptamers can be sequenced to identify the sequence and optionally a count for each aptamer. Optionally, the non-binding aptamers are eluted from the beads and are amplified by enzymatic or chemical processes.

In some instances, once the non-binding aptamers are separated and amplified, the non-binding aptamers can be sequenced to identify the sequence and optionally a count for each non-binding aptamer. The count of non-binding aptamers can provide information on which aptamers have the weakest (or strongest) binding affinity to the target relative to other aptamers in the bead-based capture system, which can supplement or validate the results of the aptamers found to bind. If aptamers are high in count for non-binding and low in count for binding, then aptamers can be determined and validated to have a weak binding affinity. If certain aptamers have significant counts for both binding and non-binding, the aptamers can be limited for some other reason (e.g., competition for binding sites among same type of aptamers).

The count can be used to determine a metric that is stored in a record in the data store or can itself be stored in a record in the data store. The record can be associated with the sequence and the record, a combination of records, and/or the data store can be associated with the particular target. Accordingly, the data store can be used to predict which sequences will code for peptides that bind with high affinity to the target. For example, a selection criterion may include identify each record associated with a count (or related metric) that is above a predefined absolute or relative threshold and is associated with the particular target. As another example, evaluation of the selection criterion may include detecting a record associated with a highest count in association with the particular target (or highest related metric) in association with the particular target. The selection criterion may be evaluated individually or in combination with one or more other criteria. Selecting a sequence may trigger an output that identifies the sequence and/or a molecule coded by the sequence.

The output may further include the count and/or an estimated binding affinity. In some instances, a selected sequence is identified as a possibility for, is recommended for, or is used for treating a given medical condition associated with the particular target.

At block 250, the sequence (or an embedded representation of the sequence), the count, and an analysis result performed based on the separation protocol (e.g., a binary classifier or a multiclass classifier) for each aptamer that has gone through the selection process of blocks 210-230 are stored in a library in a data entry. Each data entry can further be associated with and/or can identify the target for which binding was assessed. In some instances, with respect to data pertaining to the particular target, the library can selectively include data pertaining to aptamers that bound to the particular target (those that formed the aptamer-target complexes). In some instances, data for non-binders (those that did not form the aptamer-target complexes) is also included in the library. The representation of the sequence can include one-hot encoding of each nucleotide in the sequence that maintains information about the order of the nucleotides in the aptamer. The representation of the sequence can additionally or alternatively include other features concerning the sequence and/or aptamer, for example, post-translational modifications, binding sites, enzyme active sites, local secondary structure, kmers or characteristics identified for specific kmers, etc. The representation of the sequence can additionally or alternatively include a string of category identifiers, with each category identifier representing a particular nucleotide.

The count stored in the library can include a count of the aptamer detected subsequent to an exposure to the target (e.g., during incubation and potentially in the presence of other aptamers). In some instances, the count includes a count of the aptamer detected subsequent to an exposure to the target in each round of selection. The analysis result can include a binary classifier such as functionally inhibited the target, functionally did not inhibit the target, bound to the target, or did not bound to the target, a multiclass classifier such as a level of functional inhibition or a gradient scale for binding affinity.

### III. PROCESS FOR PREDICTING A SEQUENCE CODING FOR A MOLECULE PREDICTED TO BIND TO A TARGET

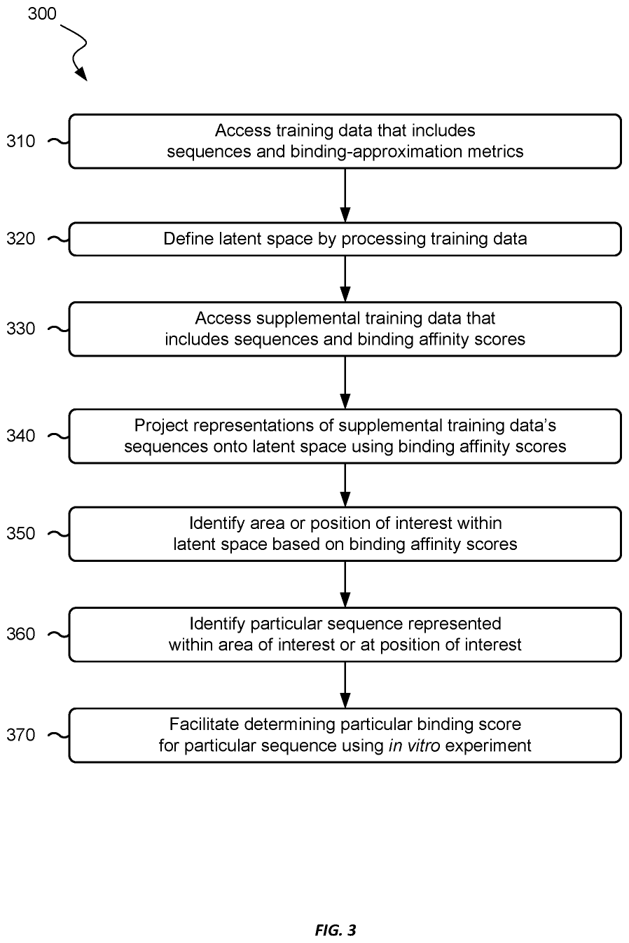

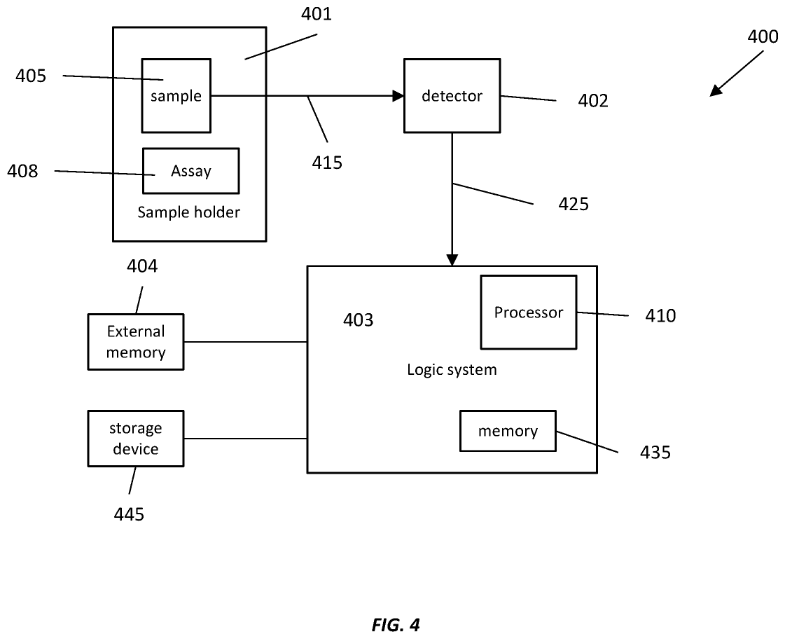

FIG. 3 is a flowchart of an example process 300 to identify a particular sequence predicted to code for a molecule to bind (e.g., at least with a threshold absolute or relative affinity) with a particular target. In some implementations, one or more process blocks of FIG. 3 can be performed by a system (e.g., system 400 of FIG. 4) and/or part or all of a network (e.g., network 100 of FIG. 1). In some implementations, one or more process blocks of FIG. 3 can be performed by another device or a group of devices separate from or including the system. Additionally, or alternatively, one or more process blocks of FIG. 3 can be performed by one or more components of computing device 500, such as processor 505, memory 510, bus 515, user input device(s) 530, display 535, and/or communications interface 540. Process 300 can include additional implementations, such as any single implementation or any combination of implementations described below and/or in connection with one or more other processes described elsewhere herein.

Process 300 begins at block 310 where latent-space controller 115 accesses training data that includes sequences and binding-approximation metrics. More specifically, the training data can include a set of training data elements. Each of the set of training data elements identifies a sequence of a molecule and a binding-approximation metric that characterizes whether the molecule binds to a particular target and/or that approximates an extent to which the molecule is more likely to bind to the particular target than other molecules associated with at least some other sequences. For example, each of the set of training data elements can include a binding-approximation metric determined by a SELEX experiment. The sequence identified by each of some or all of the training data elements can be a TNA sequence and/or can code for an aptamer. The training data can include space-defining training data 110 and can be accessed by requesting or retrieving the training data from binding-approximation collection system 105.

At block 320, latent-space controller 115 defines a latent space by processing the training data. The latent space can be defined using a machine-learning model, such as a deep neural network, a neural network with one or more skip connections, and/or a ResNet model. The latent space can be defined using a Generator model that was trained as part of a GAN or Variational Encoder model. The latent space can be defined based on part or all of a GAN or Varational Encoder model. The latent space can include at least at least 2 dimensions, at least 3 dimensions, at least 5 dimensions, at least 7 dimensions, at least 10 dimensions, at least 12 dimensions, at least 15 dimensions, at least 17 dimensions, or at least 20 dimensions. Within the latent space, each sequence can be represented via variables in at least 2 dimensions, at least 3 dimensions, at least 4 dimensions, at least 6 dimensions, at least 8 dimensions, at least 11 dimensions, at least 13 dimensions, at least 16 dimensions, at least 18 dimensions, or at least 21 dimensions. Defining the latent space can be based on representations of the sequences in the training data and also based on the binding-approximation metrics in the training data. For example, the latent space can be based on one-hot encodings of the sequences in the training data and also results indicating an affinity that SELEX experiments determined that molecules encoded by the sequences bound to the particular target.

At block 330, latent-space controller 115 accesses supplemental training data that includes sequences and binding affinity scores. More specifically, the training data elements can include a set of supplemental training data elements. Each of the set of supplemental training data elements identifies a sequence of a molecule and a binding affinity score that quantifies a strength of a binding interaction between the molecule and the particular target. For example, each of the set of supplemental training data elements can include a binding affinity score determined based on a BLI experiment. The sequence identified by each of some or all of the supplemental training data elements can be a TNA sequence and/or can code for an aptamer. The supplemental training data can include space-traversing training data 120 and can be access by requesting or retrieving the supplemental training data from binding affinity score collection system 125.

The supplemental training data accessed at block 330 can be from a different source, can have a higher accuracy, and/or can have a higher precision relative to the source, accuracy, and/or precision of the training data accessed at block 310. For example, the binding-approximation metrics in the training data can include a binary value, while the binding affinity scores in the supplemental training data can include a categorical or numeric value. As another example, the binding-approximation metrics in the training data can include a categorical value (identifying a category within a first set of categories), while the binding affinity scores in the supplemental training data can include a categorical (identifying a category within a second set of categories, where there are more categories in the second set relative to the first set) or numeric value. As yet example, the binding-approximation metrics in the training data can include a numeric value with a first number of significant figures, while the binding affinity scores in the supplemental training data can include a numeric value with more significant figurers than the first number of significant figures.

At block 340, latent-space controller 115 projects representations of sequences in the supplemental training data onto the latent space using the binding affinity scores.

At block 350, prediction generator 140 identifies an area of interest or a position of interest within the latent space based on the binding affinity scores. The area of interest or position of interest can also have been identified based on the positions of the projected representations of the sequences represented in the supplemental training data within the latent space. Prediction generator 140 can use space-traversing machine learning model 150 to identify the area or position of interest.

Identifying the area of interest can include identifying a starting position within the latent space; and identifying a direction from the starting point associated with a gradient in binding affinity scores. The area of interest within the latent space or the position of interest within the latent space can be identified using the starting position and the direction associated with the gradient.

Identifying the area of interest can include defining a separating hyperplane using another machine-learning model. The hyperplane can be configured to separate a first subset of sequence representations corresponding to aptamers that bound to the particular target from a second subset of sequence representations corresponding to aptamers that did not bind to the particular target. The hyperplane can be configured to separate a first subset of sequence representations corresponding to aptamers that bound to the particular target with at least a threshold binding affinity (or at least a threshold relative or absolute binding strength) from a second subset of sequence representations corresponding to aptamers that did not bind to the particular target with at least the threshold binding affinity (or at least the threshold relative or absolute binding strength).

At block 360, prediction generator 140 identifies a particular sequence represented within the area of interest or at the position of interest. The particular sequence can include a threose nucleic acid sequence and/or can code for an aptamer.

At block 370, downstream testing system 170 facilitates determining a particular binding score for the particular sequence using an in vitro experiment.

It will be appreciated that variations of process 300 are contemplated. For example, subsequent to block 360, another binding affinity score corresponding to the particular sequence (or even to multiple particular sequences of interest with representations within one or more areas of interest or with representations at multiple positions of interest) can be requested, accessed, and/or received for the particular sequence(s). Blocks 330-360 can then be repeated one or more times (e.g., until iteration controller 160 determines that iteration-concluding condition 165 has been satisfied), where the supplemental training data corresponds to the last iteration's particular sequence(s) of interest and one or more new particular sequences are identified for each iteration. Thus, across iterations, the data set with binding affinity scores can grow, which can facilitate accurately identifying new areas or positions of interest (e.g., by facilitating traversal of the space). As another example, subsequent to block 370, some or all of blocks 330-370 (e.g., 340-370) can be repeated one or more times.

### IV. EXAMPLE SYSTEMS

FIG. 4 illustrates a measurement system 400 according to some embodiments of the present invention. Each of one or more of binding-approximation metric collection system 105, binding affinity score collection system, and downstream testing system 170 from network 100 can include part or all of measurement system 400 and can perform some or all of the functions described in association with FIG. 4.

The system as shown includes a sample 405, such as DNA molecules within a sample holder 401, where sample 405 can be contacted with an assay 408 to provide a signal of a physical characteristic 415. An example of a sample holder can be a microfluidic flow cell that includes probes and/or primers of an assay or a tube through which a droplet moves (with the droplet including the assay). The droplet can include the aptamer families. Physical characteristic 415 (e.g., a fluorescence intensity, a voltage, or a current), from the sample is detected by detector 420. Detector 402 can take a measurement at intervals (e.g., periodic intervals) to obtain data points that make up a data signal. In one embodiment, an analog-to-digital converter converts an analog signal from the detector into digital form at a plurality of times. Sample holder 401 and detector 402 can form an assay device, e.g., a sequencing device that performs sequencing according to embodiments described herein or a fluorescence measurement device that measures fluorescent intensities associated with bound aptamers. A data signal 425 is sent from detector 402 to logic system 403. Data signal 425 can be stored in a local memory 435, an external memory 404, or a storage device 445.

Logic system 403 can be, or can include, a computer system, ASIC, microprocessor, etc. It can also include or be coupled with a display (e.g., monitor, LED display, etc.) and a user input device (e.g., mouse, keyboard, buttons, etc.). Logic system 403 and the other components can be part of a stand-alone or network connected computer system, or they can be directly attached to or incorporated in a device (e.g., a microfluidic flow cell analysis device) that includes detector 402 and/or sample holder 401. Logic system 403 can also include software that executes in a processor 410. Logic system 403 can include a computer readable medium storing instructions for controlling system 400 to perform any of the methods described herein. For example, logic system 403 can provide commands to a system that includes sample holder 401 such that sequencing or other physical operations are performed. Such physical operations can be performed in a particular order, e.g., with reagents being added and removed in a particular order. Such physical operations can be performed by a robotics system, e.g., including a robotic arm, as can be used to obtain a sample and perform an assay.

A microfluidic flow cell can be any device suitable for studying binding affinity between aptamers and tumor cells. The microfluidic flow cell can include a substrate. The volume of liquid involved in a microfluidic flow cell can be less than 1 mL, less than 1 less than 1 nL, less than 1 pL, or between any two of these stated volumes. The microfluidic flow cell can include one portion where the tumor cells are affixed. A sample of aptamers can be introduced to an inlet. The inlet can lead to a channel or a series of channels, which lead to the area where the tumor cells are affixed. Downstream of the tumor cells can be a second channel or a second plurality of channels leading to an outlet. Analysis (e.g., detection or measurement) can occur at the outlet.

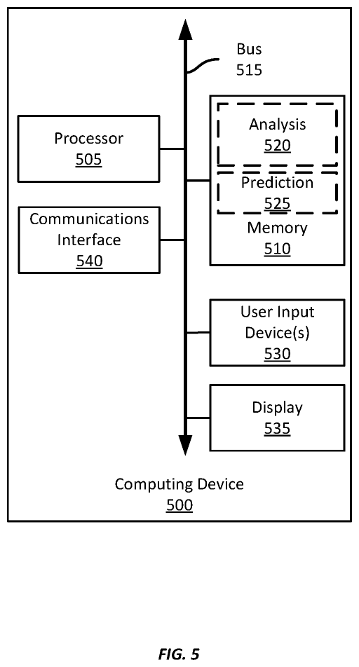

FIG. 5 illustrates an example computing device 500 suitable for use with systems and methods for identifying a particular sequence predicted to code for a molecule that will bind with a particular target according to this disclosure. Any of the computer systems mentioned herein can utilize any suitable number of subsytems, including those shown in FIG. 5. The example computing device 500 includes a processor 505 which is in communication with the memory 510 and other components of the computing device 500 using one or more communications buses 515. The processor 505 is configured to execute processor-executable instructions stored in the memory 510 to perform one or more methods for determining one or more aptamer families that characterize the one or more unknown tumor subtypes of cells, such as part or all of the example process 300 described above. In this example, the memory 510 stores processor-executable instructions that provide sequence data analysis 520 and aptamer sequence prediction 525, as discussed above with respect to FIGS. 1A, 1B, 2, and 3. Processor 505 can be processor 410. Memory 510 can be can be memory 435.

The computing device 500, in this example, also includes one or more user input devices 530, such as a keyboard, mouse, touchscreen, microphone, etc., to accept user input. The computing device 500 also includes a display 535 to provide visual output to a user such as a user interface. The computing device 500 also includes a communications interface 540. In some examples, the communications interface 540 can enable communications using one or more networks, including a local area network (“LAN”); wide area network (“WAN”), such as the Internet; metropolitan area network (“MAN”); point-to-point or peer-to-peer connection; etc. Communication with other devices can be accomplished using any suitable networking protocol. For example, one suitable networking protocol can include the Internet Protocol (“IP”), Transmission Control Protocol (“TCP”), User Datagram Protocol (“UDP”), or combinations thereof, such as TCP/IP or UDP/IP.

### V. ADDITIONAL CONSIDERATIONS

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” or “computer-readable medium” includes, but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges can independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a method” includes a plurality of such methods and reference to “the particle” includes reference to one or more particles and equivalents thereof known to those skilled in the art, and so forth. The invention has now been described in detail for the purposes of clarity and understanding. However, it will be appreciated that certain changes and modifications can be practice within the scope of the appended claims.

All publications, patents, and patent applications cited herein are hereby incorporated by reference in their entirety for all purposes. None is admitted to be prior art.

What is claimed is:

1. A computer-implemented method comprising:
accessing training data that includes a set of training data elements, each of the set of training data elements identifies a sequence of a molecule and a binding-approximation metric that characterizes whether the molecule binds to a particular target and/or that approximates an extent to which the molecule is more likely to bind to the particular target than other molecules associated with at least some other sequences;
defining a latent space for representing sequences by processing the training data using a machine-learning model;
accessing supplemental training data that includes a set of supplemental training elements, each of the set of supplemental training data elements identifying a sequence of a molecule and a binding affinity score that quantifies a strength of a binding interaction between the molecule and the particular target;
projecting representations of the sequences in the supplemental training data in the latent space using the binding affinity scores to generate an updated latent space;
identifying an area of interest within the latent space or a position of interest within the latent space based on binding affinity scores of the supplemental training data and positions of the projected representations of the sequences represented in the supplemental training data within the latent space;
identifying a particular sequence represented within the area of interest or at the position of interest; and
facilitating determining a particular binding affinity score of the particular sequence with the particular target using an in vitro experiment.

2. The computer-implemented method of claim 1, wherein identifying the area of interest within the latent space or the position of interest within the latent space includes:
identifying a starting position within the latent space; and
identifying a direction from the starting point associated with a gradient in binding affinity scores;
wherein the area of interest within the latent space or the position of interest within the latent space is identified using the starting position and the direction.

3. The computer-implemented method of claim 1, wherein the particular sequence is a threose nucleic acid sequence.

4. The computer-implemented method of claim 1, wherein identifying the area of interest within the latent space or the position of interest within the latent space includes defining a separating hyperplane using another machine-learning model.

5. The computer-implemented method of claim 1, further comprising performing a set of operations including:
projecting a representation of the particular sequence in the updated latent space using the particular binding affinity;
identifying a new area of interest within the latent space or a new position of interest within the latent space based on the particular binding affinity score and a position of the projected representation of the particular location in the latent space;
identifying a different particular sequence represented within the new area of interest or at the new position of interest; and
facilitating determining a new particular binding affinity score for the different particular sequence and the particular target using an in vitro experiment.

6. The computer-implemented method of claim 1, wherein the machine-learning model includes a generator model.

7. The computer-implemented method of claim 1, wherein the machine-learning model includes a variational autoencoder model.

8. The computer-implemented method of claim 1, wherein the machine-learning model includes a ResNet model.

9. The computer-implemented method of claim 1, wherein the latent space includes at least 12 dimensions.

10. The computer-implemented method of claim 1, wherein defining the latent space is based on the sequences of the molecules and the binding-approximation metrics in the training data.

11. The computer-implemented method of claim 1, wherein the training data was generated using a Systematic Evolution of Ligands by Exponential Enrichment (SELEX) technique.

12. The computer-implemented method of claim 1, wherein the supplemental training data was generated using a Biolayer Inferometry Analysis (BIO) technique.

13. The computer-implemented method of claim 1, further comprising:
updating a data store to store the particular binding affinity score in association with the particular sequence, wherein the data store associates various molecules with corresponding binding affinity scores that characterize binding affinities with the particular target.

14. The computer-implemented method of claim 1, further comprising:
selecting a specific sequence using the data store, wherein a selection condition is satisfied for the specific sequence; and
outputting an identification corresponding to the specific sequence.

15. The computer-implemented method of claim 1, further comprising:
outputting an identification of a potential treatment for a given medical condition or for a subject with the given medical condition, wherein the potential treatment includes molecules coded by the particular sequence.

16. A system comprising:
one or more data processors; and
a non-transitory computer readable storage medium containing instructions which, when executed on the one or more data processors, cause the one or more data processors to perform a set of actions including:
accessing training data that includes a set of training data elements, each of the set of training data elements identifies a sequence of a molecule and a binding-approximation metric that characterizes whether the molecule binds to a particular target and/or that approximates an extent to which the molecule is more likely to bind to the particular target than other molecules associated with at least some other sequences;
defining a latent space for representing sequences by processing the training data using a machine-learning model;
accessing supplemental training data that includes a set of supplemental training elements, each of the set of supplemental training data elements identifying a sequence of a molecule and a binding affinity score that quantifies a strength of a binding interaction between the molecule and the particular target;
projecting representations of the sequences in the supplemental training data in the latent space using the binding affinity scores to generate an updated latent space;
identifying an area of interest within the latent space or a position of interest within the latent space based on binding affinity scores of the supplemental training data and positions of the projected representations of the sequences represented in the supplemental training data within the latent space;
identifying a particular sequence represented within the area of interest or at the position of interest; and
facilitating determining a particular binding affinity score of the particular sequence with the particular target using an in vitro experiment.

17. The system of claim 16, wherein identifying the area of interest within the latent space or the position of interest within the latent space includes:
identifying a starting position within the latent space; and
identifying a direction from the starting point associated with a gradient in binding affinity scores;
wherein the area of interest within the latent space or the position of interest within the latent space is identified using the starting position and the direction.

18. The system of claim 16, wherein the particular sequence is a threose nucleic acid sequence.

19. The system of claim 16, wherein identifying the area of interest within the latent space or the position of interest within the latent space includes defining a separating hyperplane using another machine-learning model.

20. A computer-program product tangibly embodied in a non-transitory machine-readable storage medium, including instructions configured to cause one or more data processors to perform a set of actions including:
accessing training data that includes a set of training data elements, each of the set of training data elements identifies a sequence of a molecule and a binding-approximation metric that characterizes whether the molecule binds to a particular target and/or that approximates an extent to which the molecule is more likely to bind to the particular target than other molecules associated with at least some other sequences;
defining a latent space for representing sequences by processing the training data using a machine-learning model;
accessing supplemental training data that includes a set of supplemental training elements, each of the set of supplemental training data elements identifying a sequence of a molecule and a binding affinity score that quantifies a strength of a binding interaction between the molecule and the particular target;
projecting representations of the sequences in the supplemental training data in the latent space using the binding affinity scores to generate an updated latent space;
identifying an area of interest within the latent space or a position of interest within the latent space based on binding affinity scores of the supplemental training data and positions of the projected representations of the sequences represented in the supplemental training data within the latent space;
identifying a particular sequence represented within the area of interest or at the position of interest; and
facilitating determining a particular binding affinity score of the particular sequence with the particular target using an in vitro experiment.
